(19) United States
(12) Patent Application Publication
(10) Pub. No.: US 2015/0231369 A1
(43) Pub. Date: Aug. 20, 2015

(54) PEELABLE SHEATH

(71) Applicant: Boston Scientific Scimed, Inc., Maple Grove, MN (US)

(72) Inventors: Jeff Gray, Sudbury, MA (US); Nathan Zamarripa, Sudbury, MA (US)

(21) Appl. No.: 14/627,437

(22) Filed: Feb. 20, 2015

Related U.S. Application Data
(60) Provisional application No. 61/942,305, filed on Feb. 20, 2014.

(51) Int. Cl. A61M 25/06; A61B 17/34

(52) U.S. Cl. CPC A61M 25/0668; A61B 17/3415; A61M 2025/0675; A61M 2025/0681

(57) ABSTRACT

Peelable sheath systems and methods for using peelable sheath systems are disclosed. The peelable sheath system may include an elongated tubular sleeve (e.g., a sheath) and a sleeve separation or removing device. The peelable sheath systems may also include a hub. The elongated tubular sleeve and the sleeve separation or removing device may be positioned about an elongated member of a medical device. Initially, the sleeve separation or removing device may be positioned on the elongated member of the medical device at a location at least partially proximal the elongated tubular sleeve. In operation, the sleeve separation or removing device may be advanced toward the elongated tubular sleeve to separate a portion of the tubular sleeve from another portion of the tubular and remove the tubular sleeve from about the medical device.

### CROSS-REFERENCE TO RELATED APPLICATIONS

This application claims priority under 35 U.S.C. §119 to U.S. Provisional Application Ser. No. 61/942,305, filed Feb. 20, 2014, the entirety of which is incorporated herein by reference.

### TECHNICAL FIELD

The disclosure is directed to medical devices for use in entering lumens and/or cavities of a body and methods of using the medical devices for use in entering lumens and/or cavities of a body. More particularly, the disclosure is directed to devices and methods for facilitating insertion of medical devices into lumens and/or cavities of a body.

### BACKGROUND

Sheaths are used in medical procedures, particularly in percutaneous medical procedures. Peelable sheaths that permit removal of the sheath after insertion of the sheath into a lumen and after a catheter has been inserted through the sheath and into the lumen are known. In one peelable sheath device, a sheath is formed of a flexible tube having a pair of separation lines arranged longitudinally along the tube to form a pair of peelable sheath portions. A pair of wings is bonded to the proximal end of the sheath to facilitate separation of the peelable sheath portions.

### SUMMARY

As disclosed herein, there is a need to provide alternative devices and methods for facilitating entry into lumens and/or cavities of a body. The disclosure is directed to several alternative designs, materials, and methods of manufacturing medical device structures, assemblies, and uses thereof.

Accordingly, in one illustrative instance, a medical device may include an elongated member, an elongated tubular sleeve, and a sleeve separation device. The elongated tubular sleeve may be positioned about the elongated member. The sleeve separation device may be positioned about the elongated member, such that the sleeve separation device may be capable of traversing along a longitudinal axis of the elongated member. The sleeve separation device may be capable of at least partially removing the elongated tubular sleeve from the elongated member as it traverses along the elongated member.

Alternatively or additionally to any of the embodiments above, further comprising:

a hub portion connected to a proximal end of the elongated member.

Alternatively or additionally to any of the embodiments above, the hub portion includes a luer fitting.

Alternatively or additionally to any of the embodiments above, wherein:
the sleeve separation device includes a first portion and a second portion; and
the first portion of the sleeve separation device engages the second portion of the sleeve separation device to slidably position the sleeve separation device about the elongated member.

Alternatively or additionally to any of the embodiments above, the first portion of the sleeve separation device connects to the second portion of the sleeve separation device with a snap connection to slidably position the sleeve separation device about the elongated member.

Alternatively or additionally to any of the embodiments above, the sleeve separation device has a polygonal cross-section taken along the longitudinal axis of the elongated member.

Alternatively or additionally to any of the embodiments above, the sleeve separation device has a triangular cross-section taken along the longitudinal axis of the elongated member.

Alternatively or additionally to any of the embodiments above, wherein:

the sleeve separation device includes a first side and a second side; and

the first side meets the second side at a line intersecting the elongated member.

Alternatively or additionally to any of the embodiments above, the elongated tubular sleeve includes a first separation feature and a second separation feature, the first separation feature and the second separation feature extend in a longitudinal direction along the elongated tubular sleeve.

Alternatively or additionally to any of the embodiments above, the sleeve separation device has an edge aligned with the first separation feature and the second separation feature.

Alternatively or additionally to any of the embodiments above, the first separation feature and the second separation feature extend from a proximal end of the elongated tubular sleeve.

Alternatively or additionally to any of the embodiments above, the elongated tubular sleeve has a length and the first separation feature and the second separation feature extend substantially a distance of the length of the elongated tubular sleeve.

Alternatively or additionally to any of the embodiments above, the length of the elongated tubular sleeve extends a full length of the elongated tubular sleeve extending from a first end of the elongated tubular sleeve to a second end of the elongated tubular sleeve.

Alternatively or additionally to any of the embodiments above, a plane connecting the first separation feature and the second separation feature extends along a diameter of the elongated tubular sleeve.

Alternatively or additionally to any of the embodiments above, the sleeve separation device separates the elongated tubular sleeve into a first portion and a second portion, the first portion and the second portion are substantially equal in size.

Alternatively or additionally to any of the embodiments above, the sleeve separation device peels the elongated tubular sleeve from the elongated member.

In another illustrative instance, a peelable sheath system for removing a sheath from about a medical device is provided. The peelable sheath system may include a sheath and a sheath remover. The sheath may be positionable about a medical device and the sheath remover may be positionable about the medical device at a position proximal the sheath. The sheath remover may be positioned about the medical device such that it may be capable of moving along the medical device in a longitudinal direction. While moving along the medical device in a longitudinal direction, the sheath remover may be capable of separating the sheath into a plurality of portions. In some instances, the medical device about which the sheath and the sheath remover is positionable is a drainage catheter.

Alternatively or additionally to any of the embodiments above, wherein:
the sheath includes one or more areas of weakness;
and an edge of the sheath remover is configured to align with the one or more areas of weakness of the sheath.

Alternatively or additionally to any of the embodiments above, the one or more areas of weakness include one or more of a score line and a perforation.

Alternatively or additionally to any of the embodiments above, an opening for receiving the medical device about which the sheath is positionable extends through the edge of the sheath remover configured to align with the one or more areas of weakness of the sheath.

Alternatively or additionally to any of the embodiments above, the sheath remover has a first portion and a second portion, the first portion and the second portion are separable to removably position the sheath remover about the medical device about which the sheath is positionable.

Alternatively or additionally to any of the embodiments above, the medical device about which the sheath is positionable is one or more of an introducer, a cannula, and a drainage catheter.

Alternatively or additionally to any of the embodiments above, wherein:

the sheath includes a first insertion lumen;

the sheath remover includes a second insertion lumen;

the system further comprises a hub with a third insertion lumen; and

the first insertion lumen, the second insertion lumen, and the third insertion lumen are capable of aligning to receive the medical device.

Alternatively or additionally to any of the embodiments above, the medical device is a drainage catheter having a hub.

In yet another illustrative instance, a method of removing an elongated sleeve from a medical device is provided. The method may include providing a sleeve separation device about a medical device. The medical device may have an elongated sleeve situated thereabout. The sleeve separation device may be advanced along the medical device in a longitudinal direction of the medical device. As the sleeve separation device is advancing along the medical device in a direction of the elongated sleeve, the sleeve separation device may at least partially separate a portion of the elongated sleeve form another portion of the elongated sleeve.

Alternatively or additionally to any of the embodiments above, further comprising:

removing the sleeve separation device from about the medical device.

Alternatively or additionally to any of the embodiments above, removing the sleeve separation device from about the medical device includes separating a first portion of the sleeve separation device from a second portion of the sleeve separation device.

Alternatively or additionally to any of the embodiments above, the medical device is one of a cannula, a catheter, and an introducer.

Alternatively or additionally to any of the embodiments above, the sleeve separation device positioned about the medical device is provided at a position on the medical device that is proximal to a proximal end of the elongated sleeve.

Alternatively or additionally to any of the embodiments above, sliding the sleeve separation device along a longitudinal direction of the medical device includes sliding the sleeve separation device in a distal direction along the medical device.

The above summary of some example embodiments is not intended to describe each disclosed embodiment or every implementation of the aspects of the disclosure.

### BRIEF DESCRIPTION OF THE DRAWINGS

The aspects of the disclosure may be more completely understood in consideration of the following detailed description of various embodiments in connection with the accompanying drawings, in which:

FIG. 1 is a schematic view of a prior art peelable sheath system positioned about a medical device;

FIG. 2 is a schematic perspective view of an illustrative peelable sheath system positioned about a medical device;

FIG. 3 is a schematic perspective view of an illustrative peelable sheath system positioned about a medical device;

FIG. 4 is a schematic perspective view of features of the illustrative peelable system of the dotted circle 4 in FIG. 2;

FIG. 5 is a schematic exploded perspective view of an illustrative peelable sheath system partially positioned about a medical device;

FIG. 6 is a schematic cross-sectional view taken at line 6-6 of the illustrative peelable sheath system in FIG. 4 viewed in the proximal direction;

FIGS. 7A-7D depict a schematic flow of an illustrative method of using an illustrative peelable sheath system; and

FIG. 8 is an illustrative schematic flow diagram of a method of using a peelable sheath system.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Any relative terms, such as first, second, third, right, left, bottom, top, etc., used herein in connection with a feature are just that and are not meant to be limiting other than to be indicative of the relative relationship of the modified feature with respect to another feature.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative instances and are not intended to limit the scope of the disclosure. Selected features of any illustrative instance may be incorporated into an additional instance unless clearly stated to the contrary.

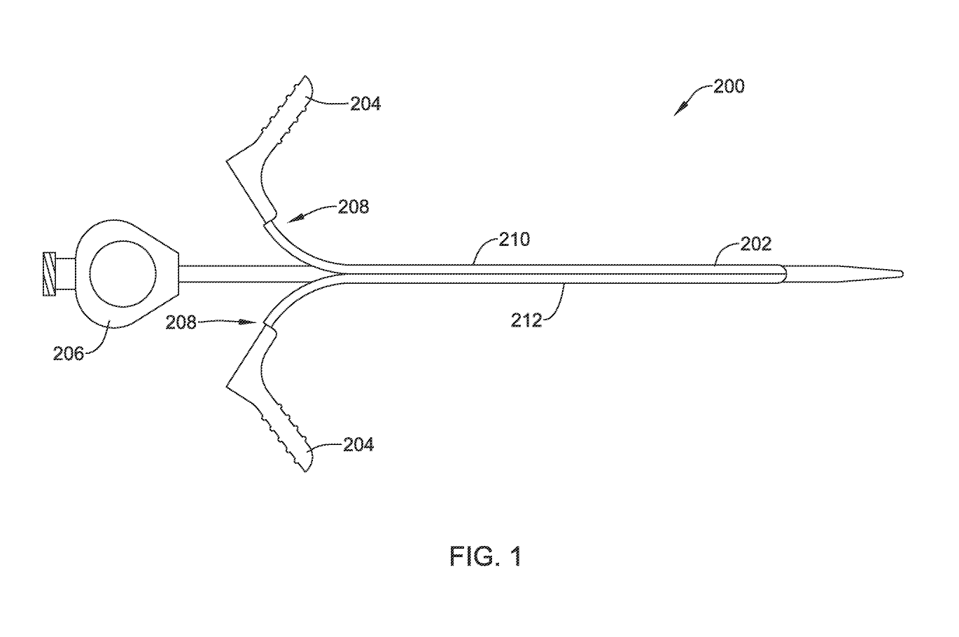

FIG. 1 shows an example peelable sheath device 200 that includes a sheath 202, a pair of wings 204, and a hub connector 206. The wings 204 may be bonded to the sheath 202 at a proximal end 208 of the sheath 202, such that each wing 204 may be attached to a different portion (e.g., a first portion 210, a second portion 212) of the sheath 202. Such arrangement of wings 204 provides longitudinal and radial points of leverage for facilitating initial separation of sheath portions 210, 212 by pulling on the wings 204 (e.g., by using one hand to pull on one wing 204 and another hand to pull on the other wing 204). After insertion of one or more medical device through the sheath 202 and insertion of the medical device and/or sheath 202 into a body lumen or cavity, the sheath 202 may be peeled away from the inserted medical device by pulling the wings 204 apart (e.g., in a radial and longitudinal direction). While peelable sheath device 200 may be effective in one or more instances, the peelable sheath device 200 requires the use of two hands to separate sheath portions 210, 212 from one another.

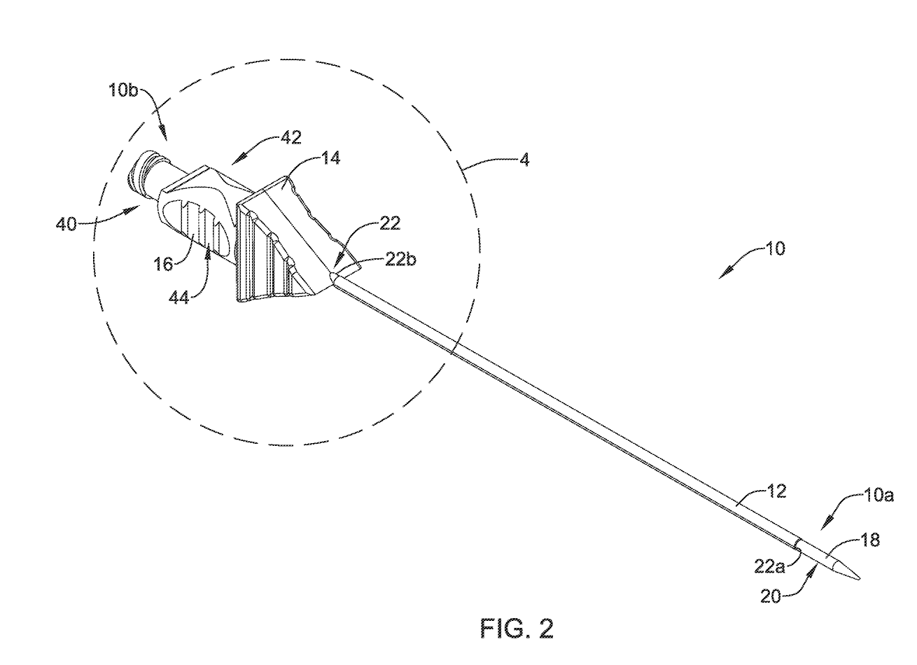

The disclosed peelable sleeve system or device 10 (e.g., a peelable sheath system or device) shown in FIG. 2 may facilitate, among other actions, actuation of a sleeve separating or removing process with a single hand or other mechanism to remove an entirety of or at least a portion of a sleeve (e.g., a sheath) from about an elongated member such as a tube or shaft (e.g., from about a medical device). Such device 10 may allow a user to utilize a second hand to secure the peelable sleeve device 10 and/or perform other tasks with the second hand. In some instances, the peelable sleeve device 10 may reduce error and/or mitigate potential complications that may occur while using other peelable sleeve or sheath devices by reducing errors and/or mitigating potential complications with air embolisms, pneumothorax, brachial plexus injuries, subclavian artery punctures, hematoma formations, sublavian vein thromboses, hemothorax, wound infections, mediastinal widening, catheter buckling, and/or other errors and complications.

The disclosed peelable sleeve device 10 may be used in percutaneous insertion of catheters in the venous or arterial system. Additionally, or alternatively, the peelable sleeve device 10 may be used in percutaneous insertion of non-venous catheters, such as non-venous drainage catheters that may, optionally, utilize a trocar needle and/or stiffening cannula for insertion into a lumen or cavity. Such uses of peelable sleeve devices 10 may provide support to the catheter and/or minimize buckling during “direct-stick” catheter placements and other catheter placements, which may be a particular concern with smaller diameter catheters (e.g., catheters with a diameter of less than eight (8) French). Alternatively, or in addition, peelable sleeves or sheaths may be used in conjunction with a stiffening cannula for added support and/or rigidity.

An illustrative peelable sleeve device 10 is depicted in FIGS. 2-7D. The peelable sleeve device 10 may include a sleeve 12 (e.g., an elongated sleeve or elongated tubular sleeve such as a sheath or other elongated tubular sleeve) and a sleeve separator 14 (e.g., a sheath separator or remover). The sleeve separator 14 may have at least one edge configured to engage the sleeve 12 and separate the sleeve 12 into portions as it engages the sleeve 12. The at least one edge may be at least sharp enough to be capable of or sufficient to split or separate the sleeve 12 into two or more portions, or may be at least thin enough to be capable of or sufficient to lift or separate the sleeve 12 into two or more portions. In some instances, the sleeve separator 14 may have one or more rounded edges to facilitate handling and/or other uses.

In some instances, the peelable sleeve device 10 may include a hub 16 and the sleeve 12, the sleeve separator 14, and/or the hub 16 may be formed in any manner and from any material. In one example, the sleeve 12, the sleeve separator 14, and/or the hub 16 may be formed using a molding technique, an extrusion technique, a forming technique, any other configuring technique, and/or any combination of configuring techniques. Illustratively, the material(s) of the sleeve 12, the sleeve separator 14, and/or the hub 16 may be any biocompatible material (e.g., a biocompatible polymer material or other biocompatible material) including, but not limited to, those materials disclosed herein, as appropriate.

The peelable sleeve device 10 may be utilized with a medical device or other ancillary device 20, where the sleeve 12 and/or the sleeve separator 14 may be positioned over (e.g., positioned about or positioned concentrically about) or configured to receive an elongated tube or shaft of the medical device or other ancillary device 20. As described further below, the sleeve separator 14 may be capable of at least partially removing or separating the sleeve 12 from about the medical device or other ancillary device 20 while the sleeve separator 14 traverses along a longitudinal axis of the medical device or other ancillary device 20. The hub 16 may be connectable with the medical device or other ancillary device 20 (e.g., at a proximal end thereof), may be a portion of the medical device or other ancillary device 20 (e.g., at a proximal end thereof), and/or may be releasably connected to the sleeve separator 14 (e.g., through a tearable connection).

Illustratively, the medical device or other ancillary device 20 may be or may include or may be used with an elongated tube or shaft. For example, the medical device or ancillary device 20 may include a needle or dilator 18, a cannula, a catheter (e.g., a drainage catheter, a delivery catheter, a diagnostic catheter, or other catheter), a guide wire, or any other medical device, where all of these devices may be, may include, or may be used with an elongated tube or shaft.

In one illustrative instance, as shown in FIG. 2, the peelable sleeve device 10 may be positioned on an elongated tube of a needle or dilator 18 (e.g., a trocar needle and a cannula) to facilitate insertion of the peelable sleeve device 10 into a lumen or cavity. One or more of the parts (e.g., the sleeve 12, the sleeve separator 14, and the hub 16) of the peelable sleeve device 10 may be separate from one another and longitudinally positioned along an axis (e.g., a longitudinal axis) of the needle or dilator 18. For example, in one instance, the sleeve separator 14 may be free from attachment to the sleeve 12 and/or the hub 16. Alternatively or in addition, one or more of the parts of the peelable sleeve device 10 may be connected to one another such that the parts of the peelable sleeve device 10 may be positioned longitudinally along an axis (e.g., a longitudinal axis) of the needle or dilator 18.

In instances when one or more of the parts of the peelable sleeve device 10 are connected to one another, the parts of the peelable sleeve device 10 may be connected in any manner. For example, the parts of the peelable sleeve device 10 may be connected with a glue, an epoxy, material of one or more of the parts of the peelable sleeve device 10, or any other connection material and/or technique. Although the parts of the peelable sleeve device 10 may be connected to one another in any manner, the connection material and/or connection technique may allow for separation of the connected parts of the peelable sleeve device 10 after insertion of the peelable sleeve device 10 into a lumen or cavity without materially affecting medical devices or other ancillary devices 20 inserted through the peelable sleeve device 10 and the cavity or lumen.

In some instances, one or more the parts of the peelable sleeve device 10 may be longitudinally positioned, such that the sleeve 12 may be positioned to extend at least partially distal the sleeve separator 14 and the hub 16, the sleeve separator 14 may be positioned at least partially proximal the sleeve 12 and at least partially distal the hub 16, and the hub 16 may be positioned at least partially proximal the sleeve 12 and the sleeve separator 14. In other instances, the parts of the peelable sleeve device 10 may have any arrangement.

In one instance, each part of the peelable sleeve device 10 may include an insertion lumen 22 (e.g., an insertion lumen 22a of the sleeve 12 (e.g., a first insertion lumen), an insertion lumen 22b of the sleeve separator 14 (e.g., a second insertion lumen), and an insertion lumen 22c of the hub 16 (e.g., a third insertion lumen)). Illustratively, the insertion lumens 22 of the parts of the peelable sleeve device 10 may be substantially aligned and configured to receive a medical device or other ancillary device 20.

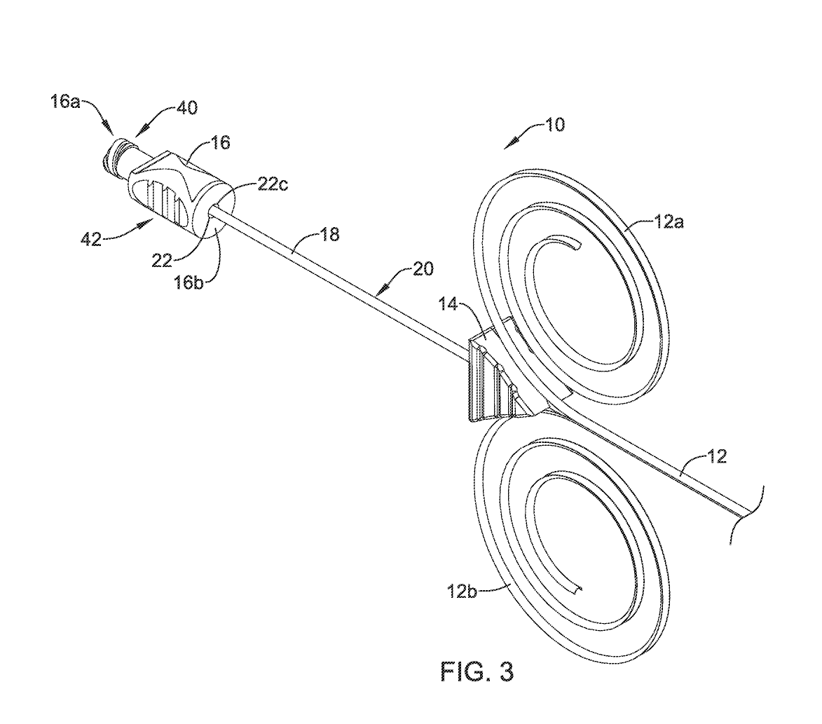

As discussed, the sleeve separator 14 may longitudinally engage the sleeve 12 to separate the sheath into one or more portions (e.g., a first portion 12a and a second portion 12b). As shown in FIG. 3, the sleeve separator 14 may be configured to slide longitudinally about a medical device or other ancillary device 20. As the sleeve separator 14 moves longitudinally in a distal direction, the sleeve separator 14 may engage the sleeve 12 and may separate the sleeve 12 into the first portion 12a and the second portion 12b.

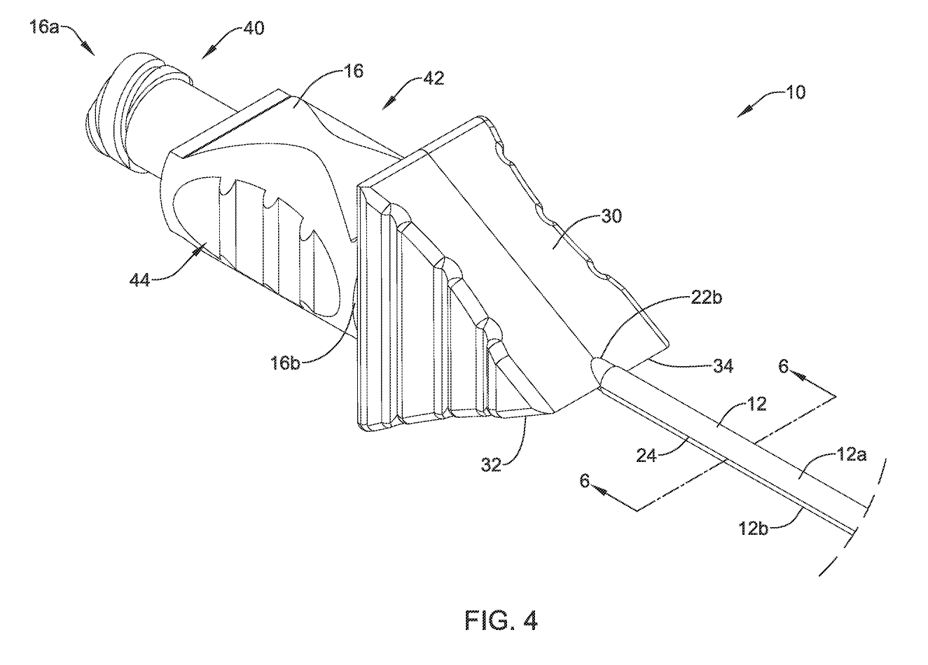

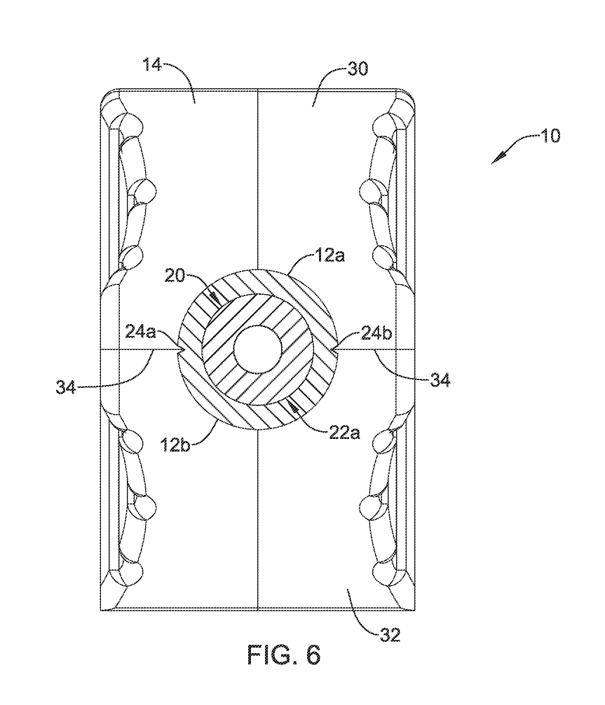

FIG. 4 is a magnification of the circle 4 in FIG. 2. As shown in FIG. 4, the sleeve 12 may include one or more areas of weakness (e.g., separation lines 24 or other separation features). The sleeve 12 may include 1 area of weakness, 2 areas of weaknesses, 3 areas of weakness, 4 areas of weakness, or more areas of weakness. In one example, the sleeve 12 may include a first separation line 24a and a second separation line 24b (see FIG. 6, as discussed below) that extend in a longitudinal direction along the sleeve. In some instances, the one or more separation lines 24 may extend generally longitudinally from a proximal end of the sleeve 12 to a distal end of the sleeve 12 (e.g., extend a distance of a length from end-to-end of the sleeve 12) or at least partially to a distal end of the sleeve 12.

The one or more separation lines 24 may be any type of separation line 24. For example, one or more of the separation lines 24 may include a score line, perforation line, any other line, or any feature facilitating separation of a first portion of the sleeve 12 from a second portion of the sleeve 12.

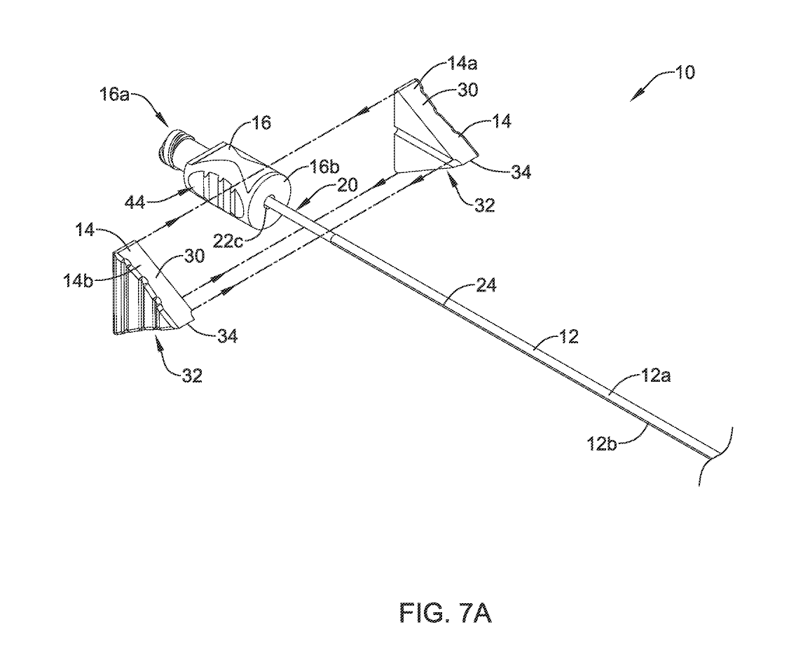

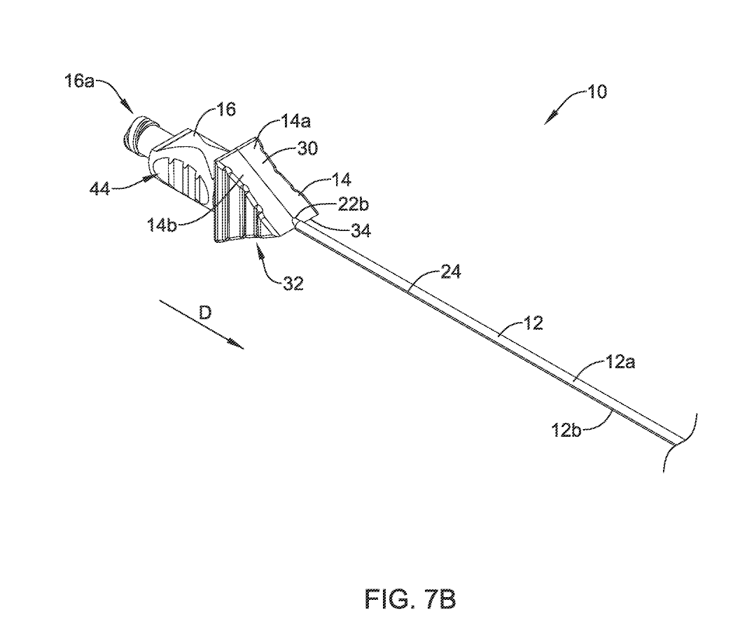

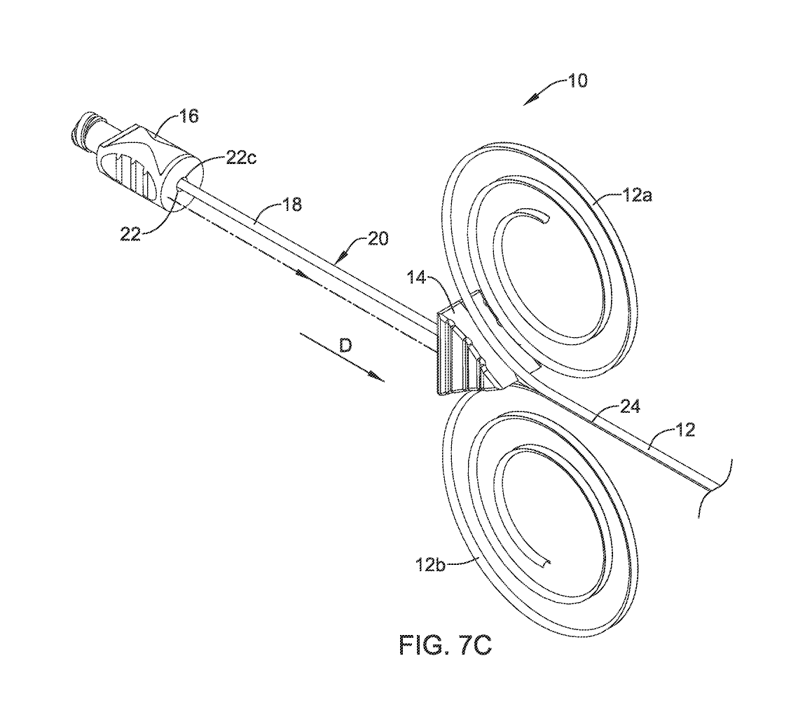

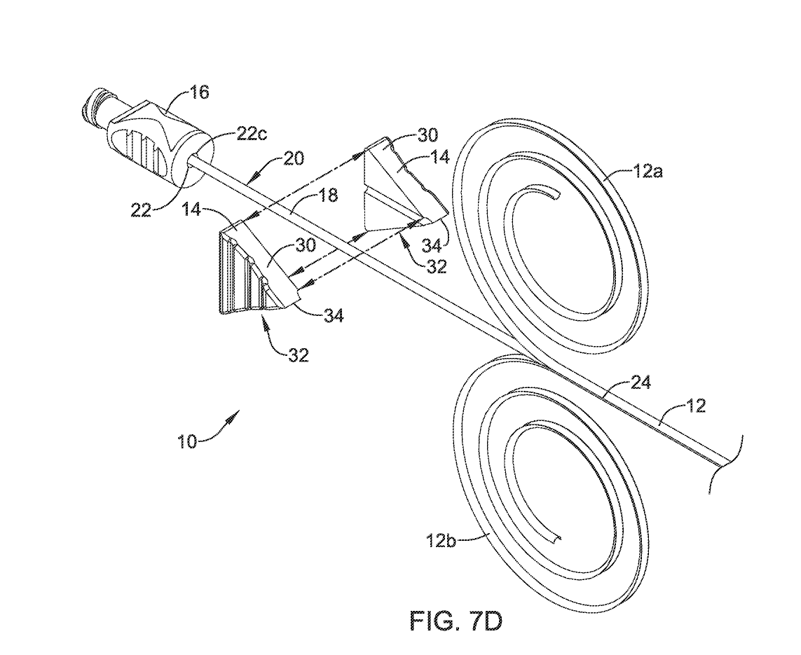

The sleeve separator 14 may take on any form capable of engaging and/or facilitating the engagement of the sleeve 12 to separate a first portion 12a from a second portion 12b of the sleeve 12. In one example, the sleeve separator 14 may have a polygonal, substantially polygonal, or other shaped cross-section and may be configured of one or more portions (e.g., see FIG. 7A for a two-portion sleeve separator 14 having triangular cross-sections). Illustratively, the sleeve separator 14 may have triangular or substantially triangular cross-section. In some instances, when the sleeve separator 14 may be positioned about the elongated tube of the medical device or other ancillary device 20 the triangular cross-section or other shaped cross-section of the sheath separator may extend in a direction along the longitudinal axis of the elongated tube.

The sleeve separator 14 may have a first side 30 and a second side 32 that meet at an edge 34 of the sleeve separator 14, where the edge 34 may align with a line intersecting the elongated tube of the medical device or other ancillary device 20 or a line intersecting an insertion lumen of the sleeve separator 14. The edge 34 of the sleeve separator 14 may align with a diameter of the insertion lumen 22b of the sheath separator and/or a diameter of the sleeve 12, where the insertion lumen 22b may extend through the edge 34.

In some instances, the edge 34 of the sleeve separator 14 may align with the first separation line 24a and the second separation line 24b of the sleeve 12. Such alignment, along with other alignments of the sleeve separator 14 and the sleeve 12, may facilitate or cause the separation of the a first portion 12a of the sleeve 12 from a second portion 12b of the sleeve 12 as the sleeve separator 14 is advanced distally in a longitudinal direction about a medical device or other ancillary device 20 inserted through the sleeve 12.

In some instances, the sleeve separator 14 may be configured to radially separate portions of the sleeve 12 from one another. For example, the first side 30 and the second side 32 of the sleeve separator 14 may extend from the edge 34 at an angle which directs separated portions of the sleeve 12 in radially outward directions from the longitudinal axis of the sleeve 12 and/or the sleeve separator 14 as the sleeve separator 14 advances distally in a longitudinal direction. The angle at which the sides 30, 32 extend from the edge 34 may be any angle between zero (0) degrees and eighty nine (89) degrees, between fifteen (15) degrees and seventy-five (75) degrees, between thirty (30) degrees and sixty (60) degrees, or forty-five (45) degrees or about forty-five (45) degrees from a plane extending through and along a longitudinal axis of a medical device or other ancillary device 20 inserted through the sleeve.

The hub 16, optionally positioned proximal the sleeve 12 and the sleeve separator 14, may include a locking portion 40 and a handle portion 42. The locking portion 40 may include a lock fitting, such as a luer lock fitting (e.g., a threaded luer lock or other luer lock fitting) or other type of lock fitting. The insertion lumen 22c of the hub 16 may extend through the locking portion 40 and the handle portion 42. In some instances, the insertion lumen 22c may extend from a first end 16a of the hub 16 to a second end 16b of the hub 16. Alternatively, the insertion lumen 22c may extend through one or more of the locking portion 40 and the handle portion 42 while extending through only a portion of a length of the hub 16 from the first end 16a to the second end 16b. The insertion lumen 22c of the hub may have a cylindrical configuration that may be in fluid communication with and/or have a similar diameter as the insertion lumen 22a of the sleeve 12 and the insertion lumen 22b of the sleeve separator 14.

In some instance, one or more objects or features may connect to the hub 16 via the locking portion 40 of the hub 16. For example, a medical device or ancillary device 20 (e.g., a hub or other portion of the medical device or ancillary device) may connect to the locking portion 40 before, during, or after insertion of the sheath into a lumen or cavity, a container capable of containing fluid may connect to the locking portion 40 to facilitate providing a fluid to or receiving fluid from a lumen or cavity in which the peelable sleeve device 10 may be inserted, and/or any other medical device or other device may connect to the locking portion 40 to facilitate passing a fluid, medical device, or other device through the insertion lumen 22. An illustrative medical device or ancillary device 20 having a hub structure that may be connected to the hub 16 of the peelable sleeve device 10 or which may have a hub structure that replaces the hub 16 of the peelable sleeve device 10 and may be used with the peelable sheath device 10 is disclosed in U.S. Patent Application Ser. No. 61/780,839 filed on Mar. 13, 2013, and entitled DRAINAGE CATHETER WITH CUTTING TOOL, which is hereby incorporated by reference in its entirety for all purpose.

The handle portion 42 of the hub 16 may be formed integrally with the locking portion 40 or may be separately formed and connected to the locking portion 40 in any manner (e.g., with a snap connection, an adhesive, or any other connector). As shown in FIGS. 2-7, the handle portion 42 may have one or more grip features 44. In one example, the grip features 44 may include indentations or raised features configured to facilitate maintaining a hold on the hub 16. In some instances, when the handle portion 42 of the hub has a plurality of sides, the grip features 44 may be positioned on one or more sides of the handle portion 42 of the hub 16. In instances when the handle portion 42 has a substantially circular cross-section, the grip features 44 may extend entirely around a circumference of the handle portion 42 and/or may extend only partially around the handle portion 42.

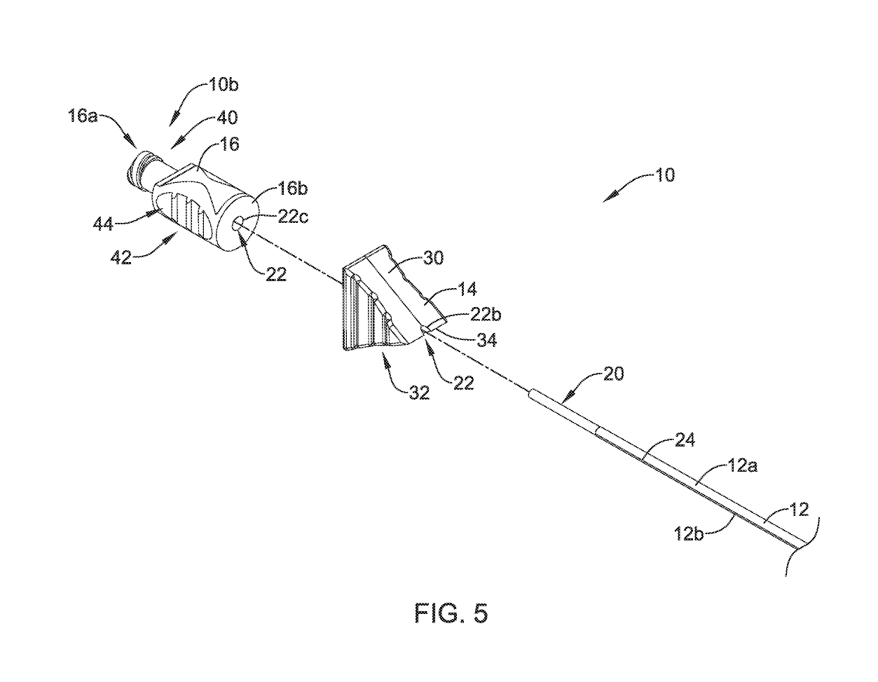

FIG. 5 is an exploded schematic perspective view of the peelable sleeve device 10. As shown in FIG. 5, the insertion lumen 22 of the peelable sleeve device 10 may include an insertion lumen 22a of the sleeve 12, an insertion lumen 22b through the sleeve separator 14, and an insertion lumen 22c through the hub 16. Illustratively, a medical device or other ancillary device 20 or a fluid may be inserted into a lumen or cavity in which the sleeve 12 is inserted through a plurality of insertion lumens 22 (e.g., insertion lumen 22a, insertion lumen 22b, and/or insertion lumen 22c) of the peelable sleeve device 10.

FIG. 6 depicts an illustrative end view of a distal end 10a of the peelable sleeve device 10 which may have a proximal end 10b at an opposite end from distal end 10a. In the distal end view of the peelable sleeve device 10, the separation lines 24 of the sleeve 12 are depicted.

In one example, as shown in FIG. 6, the sleeve 12 may include a first separation line 24a and a second separation line 24b. In some instances, the separation lines 24a, 24b may be utilized to at least partially define the first portion 12a and the second portion 12b of the sleeve 12. For example, the separation lines 24a and 24b may be configured such that a plane extending through the first separation line 24a and the second separation line 24b may hemispherically dissect the sleeve 12 along a diameter thereof, as shown in FIG. 6. Alternatively, the separation lines 24 or other separating features may facilitate separating the sleeve 12 into two or more portions of any size and/or configuration.

As discussed above the separation lines 24 may be any type of line or feature that may be capable of facilitating separation of a first portion 12a of the sleeve 12 from a second portion 12b of the sleeve 12 (e.g., separation of the first portion 12a of the sleeve 12 from the second portion 12b such that each portion 12a, 12b of the sleeve 12 has substantially the same size and/or dimensions or different sizes and/or dimensions). For example, one or more of the separation lines 24 may include a perforation in the sleeve 12, an indentation in the sleeve 12, a seam in the sleeve 12, a feature embedded in the sleeve 12, a continuous line in the sleeve 12, a discontinuous line in the sleeve 12, one or more holes or openings in the sleeve 12, scoring in the sleeve 12, etching in the sleeve 12, lines of weakness in the sleeve 12, thinning in the sleeve 12, weaker material seam, and/or any other feature of, in, or interacting with the sleeve 12 capable of facilitating the separation of the first portion 12a of the sleeve 12 from the second portion 12b of the sleeve 12. The separation lines 24 or separation features may be scored or formed as part of an extrusion or secondary process or may be formed in any other manner.

In some instances, the edge 34 where the first side 30 and the second side 32 meet at a distal end of the sleeve separator 14, or any other edge at the distal end of the sleeve separator 14, may align with one or more separation lines 24. For example, as shown in FIG. 6, the edge 34 of the sleeve separator 14 may align with two separation lines (e.g., the first separation line 24a and the second separation line 24b) to facilitate separation of the first portion 12a of the sleeve 12 from the second portion 12b of the sleeve 12 as the sleeve separator 14 is advanced in a distal direction along a medical device inserted through the sleeve separator 14 and/or the sleeve 12.

In some cases, the sleeve separator 14 may rotate with the hub 16 and/or the sleeve 12 about a medical device or ancillary device 20 extending therethough when the sleeve separator 14, the hub 16 are connected. Additionally, or alternatively, the sleeve 12, the sleeve separator 14, and/or the hub 16 may be freely rotatable about a medical device extending therethrough when the sleeve separator 14 is separated from the hub 16 and the sleeve 12. In some instances, the sleeve 12 may have one or more features at a proximal end of the sleeve 12 configured to receive the edge 34 of the sleeve separator 14, where the one or more features at the proximal end of the sleeve 12 may be capable of aligning the edge at a distal end of the sleeve separator 14 (e.g., edge 34) with the one or more separation lines 24. In one example, the one or more features at the proximal end of the sleeve 12 may be a notch, an indentation, a stop, an area of friction and/or other features capable of receiving the edge 34 at a distal end of the sleeve separator 14. Illustratively, the features at the proximal end of the sleeve 12 may include one or more of a V-shaped, U-shaped, C-shaped or other shaped notch, indentation, flap, or area of friction and/or any other similar or dissimilar feature capable of aligning or capable of facilitating alignment with the edge 34 at a distal end of the sleeve separator 14 with the one or more separation lines 24 of the sleeve 12.

The sleeve separator 14 may be comprised of one or more portions. For example, as shown in FIG. 7A, the sleeve separator 14 may include a first portion 14a and a second portion 14b. Two or more portions of the sleeve separator 14 may engage one another at any position to form the sleeve separator 14. In one example, the first portion 14a of the sleeve separator 14 and the second portion 14b of the sleeve separator 14 may engage one another about a plane extending along a longitudinal axis of the insertion lumen 22a of the sleeve separator 14 to facilitate slidably positioning the sleeve separator 14 about the elongated tube of the medical device or other ancillary device 20. Such a configuration of the sleeve separator 14 having multiple portions may facilitate positioning the sleeve separator 14 about a medical device or other ancillary device 20 without the need to insert one or more of a proximal end and a distal end of the medical device or ancillary device 20 through or into the insertion lumen 22a of the sheath separator. Additionally, or alternatively, a sleeve separator 14 having a plurality of separable and engaging portions may facilitate adding the sheath separator to a medical device or other ancillary device 20 that has a hub thereon or connected thereto and/or has been inserted through the insertion lumen 22a of the sleeve 12.

The portions (e.g., the first portion 14a and the second portion 14b) of the sleeve separator 14 may engage one another or may be connected to one another in any manner and in any form. For example, the portions of the sleeve separator 14 may engage one another via a magnetic connection, a snap connection, a latch connection, a lock and key connection, any other engaging or connecting technique, and/or any combination thereof.

FIGS. 7A-7D depict a method of removing the sleeve 12 from about a medical device or other ancillary device 20, where the medical device or other ancillary device 20 may be connected to or inserted through the hub 16, inserted at least partially through the sleeve 12 (e.g., sheath or other elongated tube). The sleeve separator 14 may be separated from the medical device or ancillary device 20 and the first portion 14a of the sleeve separator 14 and the second portion 14b of the sleeve separator 14 may be separated from one another for positioning about the medical device or other ancillary device 20 between the hub 16 and the sleeve 12, as shown in FIG. 7A. Once the medical device or other ancillary device 20 has been inserted into a cavity or lumen, or at any other time, the first portion 14a and the second portion 14b of the sleeve separator 14 may be positioned and connected (see dotted lines with arrows in FIG. 7A) to form the insertion lumen 22b of the sleeve separator 14 about the medical device or other ancillary device 20, as shown in FIG. 7B. After engaging the portions of the sleeve separator 14 to one another, the edge 34 of the sleeve separator 14 may be aligned with the separation lines 24 of the sleeve 12.

As shown in FIG. 7C, the sleeve separator 14 may then be manually or otherwise advanced or slid in a distal direction D (see dotted line with arrow indicating movement of the sleeve separator 14) about the medical device or other ancillary device 20 to engage the proximal end of the sleeve 12 and separate and/or split the sleeve 12 into the first portion 12a and the second portion 12b as the sleeve separator 14 is advanced toward the distal end of the sleeve 12, as shown in FIG. 7C. In some instances, for example, when the hub 16 is not attached to another device or apparatus, a distal end of the hub 16 may abut the proximal end of the sleeve separator 14, such that the hub 16 may be advanced distally to advance the sleeve separator 14 in the distal direction D and cause separation and/or removal of the sleeve 12 from the medical device or other ancillary device 20.

Once the sleeve 12 has been fully or at least partially split, separated, and/or peeled away from the medical device or other ancillary device 20, the sleeve separator 14 may then be removed from the medical device or ancillary device 20. Illustratively, the first portion 14a of the sleeve separator 14 positioned about the medical device or ancillary device 20 may be manually or automatically separated from the second portion 14b (see arrows in FIG. 7D indicating separation of the sleeve separator 14). The first portion 14a and the second portion 14b may be manually or automatically separated from one another in any manner (e.g., releasing the engaging technique discussed above by reversing the engaging technique and/or by force) and removed from the medical device or other ancillary device 20, as shown in FIG. 7D. In one illustrative instance, the first portion 14a of the sleeve separator 14 may be automatically separated from the second portion 14b of the sleeve separator 14 when it engages a separating portion or feature (not shown) of the sleeve 12 and/or the medical device or ancillary device 20 (e.g., an outer diameter feature of the sleeve 12 or medical device or other ancillary device 20 that is greater than the diameter of the insertion lumen 22b of the sleeve separator 14 as the sleeve separator 14 advances in the distal direction D).

In some cases, the first portion 14a and the second portion 14b of the sleeve separator 14 may be separated from one another such that one portion 14a, 14b may remain along the medical device or ancillary device 20 and the other portion 14a, 14b may separate from the portion remaining along the medical device or ancillary device. In such cases, the insertion lumen 22b or a portion of the insertion lumen 22b of the sleeve separator 14 (e.g., of the portion 14a, 14b of the sleeve separator 14 remaining along the medical device or ancillary device 20) may remain along or about the medical device or ancillary device 20 while the separated portion of the sleeve separator 14 may be removed from a position along the medical device or ancillary device 20.

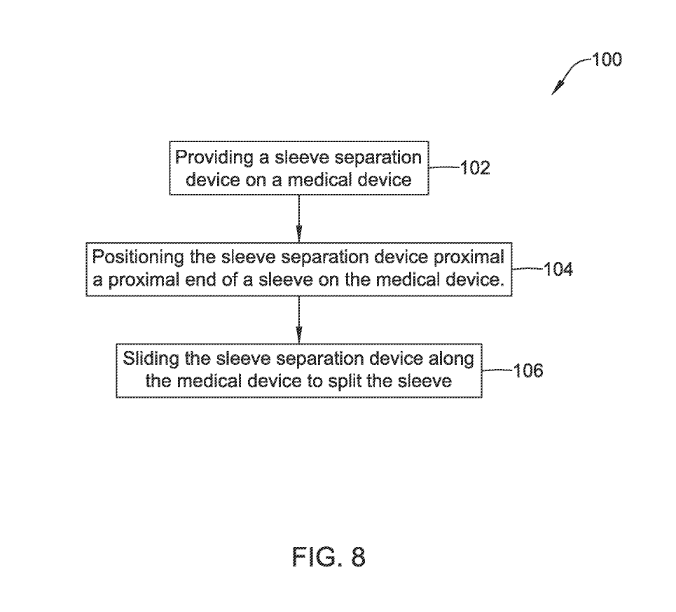

FIG. 8 shows a flow diagram for a method 100 of using the peelable sleeve device 10 having, among other features, a sheath or sleeve 12 and a sleeve separator 14 (e.g., a sleeve separation or removal device). In the method 100 using the peelable sleeve device 10 on a medical device or other ancillary device 20 (e.g., a catheter, a needle or dilator 18, a drainage catheter, etc.), the medical device or other ancillary device 20 may be inserted through the sleeve 12 and the peelable sleeve device 10 on the medical device or other ancillary device 20 may be provided 102 to a user.

The sleeve separator 14 of the peelable sleeve device 10 may be positioned on and/or about the medical device or other ancillary device 20. Illustratively, the sleeve separator 14 may be positioned 104 on and/or about the medical device or other ancillary device 20 at a position proximal a proximal end of the sleeve 12. When the sleeve separator 14 is positioned about the medical device or other ancillary device 20 and at least partially proximal the proximal end of the sleeve 12, or at other times and/or other configurations, the sleeve separator 14 may be advanced (e.g., slid or otherwise moved) 106 along the medical device or other ancillary device 20 in a distal and/or longitudinal direction (e.g., toward a distal end of the sleeve 12 or medical device or other ancillary device 20) to separate and/or split a portion of the sleeve 12 (e.g., a first portion 12a of the sleeve 12) from another portion of the sleeve 12 (e.g., a second portion 12b of the sleeve 12) and/or from or about the medical device or other ancillary device 20. Once the sleeve 12 has been separated into one or more portions, the sleeve 12 may be removed from about the medical device or other ancillary device 20.

In one illustrative instance, the sleeve 12 of the peelable sleeve device 10 may be provided on a needle or dilator 18 and the sleeve 12 along with the needle or dilator 18 may be inserted into a lumen or cavity (e.g., a body lumen or body cavity of a patient) (not shown). After insertion of the needle or dilator 18 and the sleeve 12 into the lumen or cavity, the needle or dilator 18) may be withdrawn or otherwise removed from the lumen or cavity and the sleeve 12. Thus, the sleeve 12 may be left protruding from the lumen or cavity to facilitate insertion of one or more devices into the lumen or cavity.

In one example, a medical device or other ancillary device 20 (e.g., a drainage catheter, a diagnostic catheter, a therapeutic catheter, guidewire, or other medical device) may be inserted into the sleeve 12 left protruding from the lumen or cavity. Once the medical device or other ancillary device 20 is inserted into the lumen or cavity through the sleeve 12, the sleeve 12 may be longitudinally sheared, peeled, or separated and removed from the inserted medical device or other ancillary device 20 and lumen or cavity.

In one illustrative example, the sleeve separator 14 may be positioned about the medical device or other ancillary device 20 at a position at least partially proximal the proximal end of the sleeve 12. Once the sleeve separator 14 is positioned, it may be advanced or slid distally and longitudinally along the medical device or other ancillary device 20 to separate, peel, or shear the sleeve 12 from around the medical device or other ancillary device 20. Once, before, or while the sleeve 12 is being separated, peeled, or sheared into portions (e.g., the first portion 12a and the second portion 12b), the sheath may be removed from the lumen or cavity and discarded.

In an illustrative instance of use with a non-venous catheter in a percutaneous insertion method, the sleeve 12 of the peelable sleeve device 10 may be positioned on and/or about a drainage catheter. Drainage catheters may be “direct-stick” catheters and positioning the sleeve 12 about the drainage catheter may provide added support (in addition to the natural strength/support of the drainage catheter) to the catheter during insertion thereof into a patient (e.g., through skin of the patient). Such positioning of the sleeve about the drainage catheter may have mitigating effects on the chances of the drainage catheter buckling during insertion. After insertion of the drainage catheter and/or the sleeve 12 into the patient, a sleeve separator 14 positioned about the drainage catheter at least partially proximal to the sleeve 12 may be advanced forward (e.g., forced forward via an abutment with a hub of the drainage catheter) as the drainage catheter is advanced into the patient. The advancement of the sleeve separator 14 may result in the sleeve separator 14 contacting the sleeve 12 and the shearing, splitting, or peeling the sleeve 12 into a plurality of portions. Once the drainage catheter reaches a target depth, the sleeve may be separated into portions such that it may be removed from about the drainage catheter and/or from insertion into the patient. The sleeve separator 14 may be removed from about the drainage catheter by separating the sleeve separator 14 into a plurality of portions (e.g., a first portion 14a and a second portion 14b) that connect to form an insertion lumen 22b extending therethrough and an edge 34 for engaging the sleeve 12.

Although particular method features may be described herein in particular orders, it is contemplated that the features of the disclosed methods may be performed in other orders and the orders presented are merely illustrative.

As discussed above, the sleeve 12, the sleeve separator 14, and/or hub 16 may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some instances the sleeve can be blended with a liquid crystal polymer (LCP). For example, the mixture can contain up to about 6 percent LCP.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A medical device, comprising:
an elongated member having a longitudinal axis;
an elongated tubular sleeve positioned about the elongated member;
a sleeve separation device positioned about the elongated member, the sleeve separation device capable of at least partially removing the elongated tubular sleeve from about the elongated member while traversing along the elongated member about the longitudinal axis of the elongated member.

2. The medical device of claim 1, further comprising:
a hub portion connected to a proximal end of the elongated member.

3. The medical device of claim 1, wherein:
the sleeve separation device includes a first portion and a second portion; and
the first portion of the sleeve separation device engages the second portion of the sleeve separation device to slidably position the sleeve separation device about the elongated member.

4. The medical device of claim 3, wherein the first portion of the sleeve separation device connects to the second portion of the sleeve separation device with a snap connection to slidably position the sleeve separation device about the elongated member.

5. The medical device of claim 1, wherein the sleeve separation device has a polygonal cross-section taken along the longitudinal axis of the elongated member.

6. The medical device of claim 1, wherein the sleeve separation device has a triangular cross-section taken along the longitudinal axis of the elongated member.

7. The medical device of claim 1, wherein:
the sleeve separation device includes a first side and a second side; and
the first side meets the second side at a line intersecting the elongated member.

8. The medical device of claim 1, wherein the elongated tubular sleeve includes a first separation feature and a second separation feature, the first separation feature and the second separation feature extend in a longitudinal direction along the elongated tubular sleeve.

9. The medical device of claim 8, wherein the sleeve separation device has an edge aligned with the first separation feature and the second separation feature.

10. The medical device of claim 8, wherein the first separation feature and the second separation feature extend from a proximal end of the elongated tubular sleeve.

11. The medical device of claim 8, wherein the elongated tubular sleeve has a length and the first separation feature and the second separation feature extend substantially a distance of the length of the elongated tubular sleeve.

12. The medical device of claim 11, wherein the length of the elongated tubular sleeve extends a full length of the elongated tubular sleeve extending from a first end of the elongated tubular sleeve to a second end of the elongated tubular sleeve.

13. The medical device of claim 8, wherein a plane connecting the first separation feature and the second separation feature extends along a diameter of the elongated tubular sleeve.

14. The medical device of claim 1, wherein the sleeve separation device peels the elongated tubular sleeve from the elongated member.

15. A peelable sheath system for removing a sheath from about a medical device, comprising:
a sheath positionable about a medical device;
a sheath remover positionable about the medical device at a position proximal the sheath; and
wherein the sheath remover is capable of moving along the medical device in a longitudinal direction and capable of separating the sheath into a plurality of portions as it moves along the medical device in the longitudinal direction.

16. The system of claim 15, wherein:
the sheath includes one or more areas of weakness;
and an edge of the sheath remover is configured to align with the one or more areas of weakness of the sheath.

17. The system of claim 16, wherein the one or more areas of weakness include one or more of a score line and a perforation.

18. The system of claim 16, wherein an opening for receiving the medical device about which the sheath is positionable extends through the edge of the sheath remover configured to align with the one or more areas of weakness of the sheath.

19. The system of claim 15, wherein:
the sheath includes a first insertion lumen;
the sheath remover includes a second insertion lumen;
the system further comprises a hub with a third insertion lumen; and
the first insertion lumen, the second insertion lumen, and the third insertion lumen are capable of aligning to receive the medical device.

20. A method of removing an elongated sleeve from a medical device, the method comprising:
advancing a sleeve separation device having an elongated sleeve thereabout along a medical device in a longitudinal direction of the medical device to at least partially separate a portion of the elongated sleeve from another portion of the elongated sleeve;
removing the sleeve separation device from about the medical device; and
wherein removing the sleeve separation device from about the medical device includes separating a first portion of the sleeve separation device from a second portion of the sleeve separation device.
